(19) United States
(12) Patent Application Publication
(10) Pub. No.: US 2009/0318892 A1
(43) Pub. Date: Dec. 24, 2009

(54) Removable Core Implant Delivery Catheter

(76) Inventors: Maria Aboytes, Palo Alto, CA (US); Frank Becking, Santa Clara, CA (US)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(21) Appl. No.: 12/143,502

(22) Filed: Jun. 20, 2008

(51) Int. Cl. A61M 25/00

(52) U.S. Cl. 604/510; 604/523; 604/527

(57) ABSTRACT

A microcatheter comprising a sleeve and removable core with an atraumatic tip that allows delivery and navigation of the catheter to a site in remote small diameter vasculature over a guidewire. The core is removed upon achieving desired vascular access, leaving the sleeve in place for delivery of various therapeutic implants or systems.

### BACKGROUND OF THE INVENTION

The design of traditional microcatheters often balances flexibility and column strength so that the microcatheter is sufficiently pliable to pass through very small turns of the vascular system when advanced into narrow and tortuous vessels. Often lumen size is sacrificed in exchange for wall thickness in order to provide a sufficiently navigatable catheter. Examples of current catheters that exemplify such compromises may be found in U.S. Pat. No. 4,739,768 to Engleson and U.S. Pat. No. 5,851,203 to van Muiden.

A need remains for a microcatheter that is able to navigate in the cerebral vasculature while maintaining a relatively large diameter working lumen without signficant wall thickness, resulting in a large outside diameter as well.

### SUMMARY OF THE INVENTION

Disclosed herein are microcatheters that provide access to remote regions in the neuro-vasculature. The microcatheters can also be used in other small diameter and/or tortuous vessels such as those found in the liver and kidneys, etc.

The microcatheters of the present invention provide a working lumen for a physician to advance various delivery systems to the remote region in the cerebral (or other) vasculature. Delivery systems for passage through the subject microcatheters include, but are not limited to balloon expandable or self expanding stents, and stent-graft systems. Advantageously, stent-grafts as described in provisional patent application U.S. Ser. No. 61/035328 filed Mar. 10, 2008, incorporated by reference in its entirety, may be delivered using the subject access system.

In one variation, the access system includes a microcatheter adapted for accessing tortuous vasculature where the microcatheter includes a tubular sleeve having a thin wall and including a proximal portion, a distal portion and a lumen extending therethrough, the distal portion terminating at a distal opening, and a core dedicated member removably located within the sleeve lumen, the core member having a proximal end, a distal end terminating in an atraumatic tip and a guidewire lumen extending therethrough. A length of the core member is typically greater than a length of the tubular sleeve and has flexibility, pushability (and possibly also torsional load bearing capacity) which permits core member navigation to the cerebral vasculature (within the sleeve and typically over the guidewire). The core member has an atraumatic tip extending distally beyond a distal opening of the tubular sleeve. The tubular sleeve comprises a length, outer diameter and flexibility to permit navigation to the cerebral vasculature typically only when the core member is located within the body lumen. Alone, the tubular member is quite flexible and weak and its walls are thin (about 0.005″ or less and as thin as about 0.003″).

Together with the core member, however, the microcatheter system can navigate through tortuous vasculature to a target site, after which the core member is removed and the sleeve lumen used for delivering prosthesis or other devices. The microcatheter system is able to achieve a working ID of about 0.050 inches or greater and still be navigatable to distal neurovascular sites.

The invention further includes methods of accessing a remote region in blood vessels of the cerebral vasculature by delivering a guidewire to a remote region of a blood vessel, navigating a microcatheter to the remote region by advancing the microcatheter over the guidewire. The microcatheter comprises a tubular sleeve having a core member located within a lumen of the tubular sleeve; and where without the core member the tubular sleeve lacks sufficient column strength to advance to the remote region.

The method further comprises locating the opening of the tubular sleeve at a target access site (e.g. in the cerebral vasculature) withdrawing the core member from the tubular sleeve while maintaining the tubular sleeve at the remote region of vasculature, advancing an implant delivery system having an implant through the tubular sleeve to the remote region of vasculature, and deploying the implant in the blood vessel. Eventually, the sleeve and guidewire (if it still remains during the implant delivery) can also be removed.

The invention includes a kit having at least the sleeve and core members, and also optionally including a guidewire over which can slide the core member to help direct the system to a target site.

### BRIEF DESCRIPTION OF THE DRAWINGS

The figures provided herein are not necessarily drawn to scale, with some components and features are exaggerated for clarity. Each of the figures diagrammatically illustrates aspects of the invention.

FIG. 1A illustrates one variation of an access system according to the present disclosure.

FIG. 1B illustrates a magnified view of a distal portion of the device of FIG. 1A with a cutaway view of its shaft.

FIG. 2A illustrates advancement of an access system to a remote region in the cerebral vasculature.

FIGS. 2B to 2D show one example of use of the subject microcatheter system to navigate to a remote site in the vasculature to create a path to the site for advancement of a subsequent therapeutic device.

FIGS. 2E to 2G show use of the system to deliver another type of therapeutic device.

FIG. 3A illustrates a cross sectional view of a distal portion of a sleeve and core member at a distal end of the access device.

FIG. 3B illustrates a core member having a plurality of slots to increase flexibility.

FIG. 3C illustrates each of another core member and a reinforced sleeve.

Variations of the invention from the embodiments pictured is contemplated. Accordingly, depiction of aspects and elements of the invention in the figures is not intended to limit the scope of the invention.

### DETAILED DESCRIPTION OF THE INVENTION

Various exemplary embodiments of the invention are described below. Reference is made to these examples in a non-limiting sense. They are provided to illustrate more broadly applicable aspects of the present invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. All such modifications are intended to be within the scope of the claims made herein.

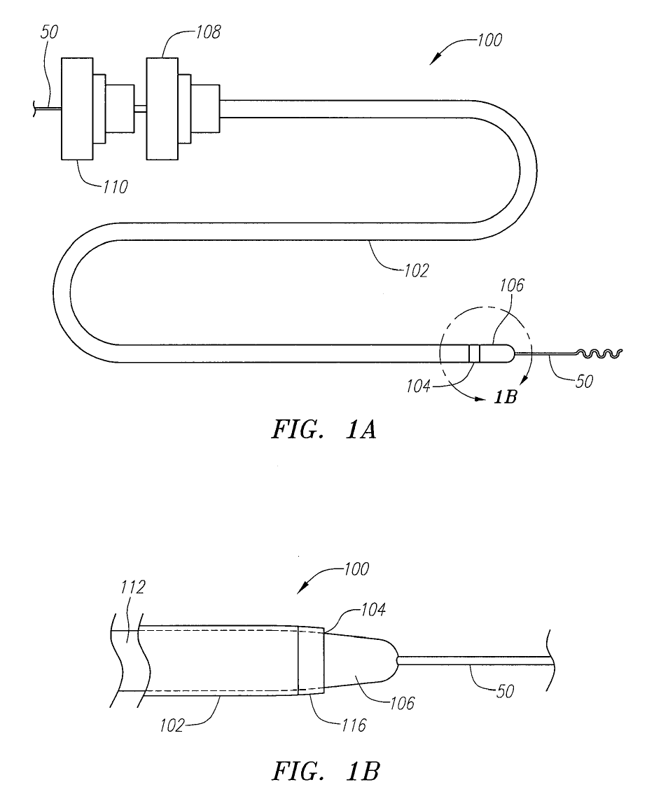

FIG. 1A illustrates a first variation of microcatheter system 100 according to the present invention. A tubular sleeve 102 has a distal opening 104 protected by an atraumatic tip 106. Atraumatic distal tip 106 is part of a core member 112 (shown in FIG. 1B) that extends through sleeve 102. System 100 is configured so that tubular sleeve 102 and core member can navigate over a wire 50, through tortuous vasculature to reach remote regions in the body, such as the vasculature of the brain. Upon arriving at the target site, core 112 can be withdrawn from sleeve 102. A physician can then deliver one or more therapeutic devices through the sleeve 102 while it remains in the blood vessel (and optionally over the guidewire if it remains within the sleeve). The sleeve may also be used for device access after removal of the guidewire.

FIG. 1A also shows tubular sleeve 102 having a sleeve hub 108 and core member 112 having a core hub 110. Hubs 108 and 110 can be any conventional type hub or body that is typically used to manipulate medical catheters. Although core hub 110 is shown as being spaced from sleeve hub 108, the system can be configured so the hubs removably nest or lock when atraumatic tip 106 is adjacent to distal opening 104 of sleeve 102. The length of the core member 112 is selected to be greater than a length of sleeve 102 to allow for the atraumatic tip to extend more distally beyond the opening of sleeve 102. Marker band 116 allows visualization of microcatheter navigation and placement. Though not shown, the core may also incorporate various radiopaque markers.

FIG. 1B shows a magnified view of the region 1B in FIG. 1A. Atraumatic tip 106 is typically bullet shaped and located at an end of core member 112 as illustrated. However, alternate tip configurations (e.g., semi-spherical, rounded cone, ball-tipped, etc.) are equally suitable.

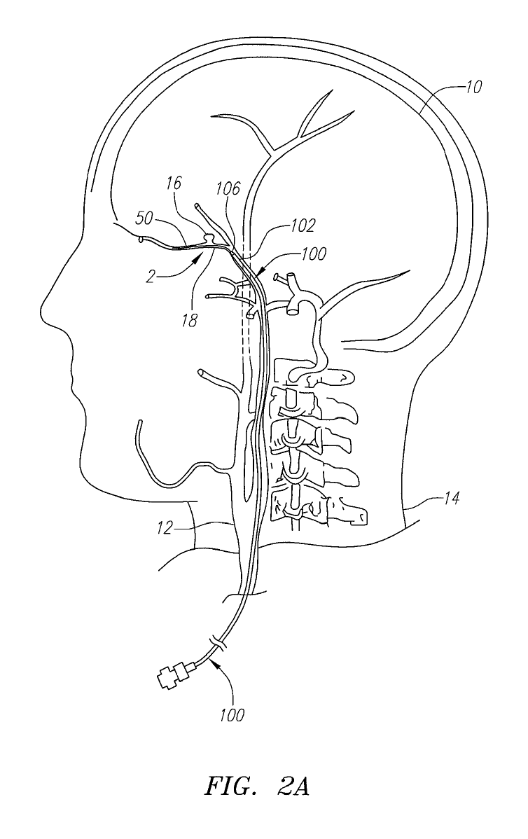

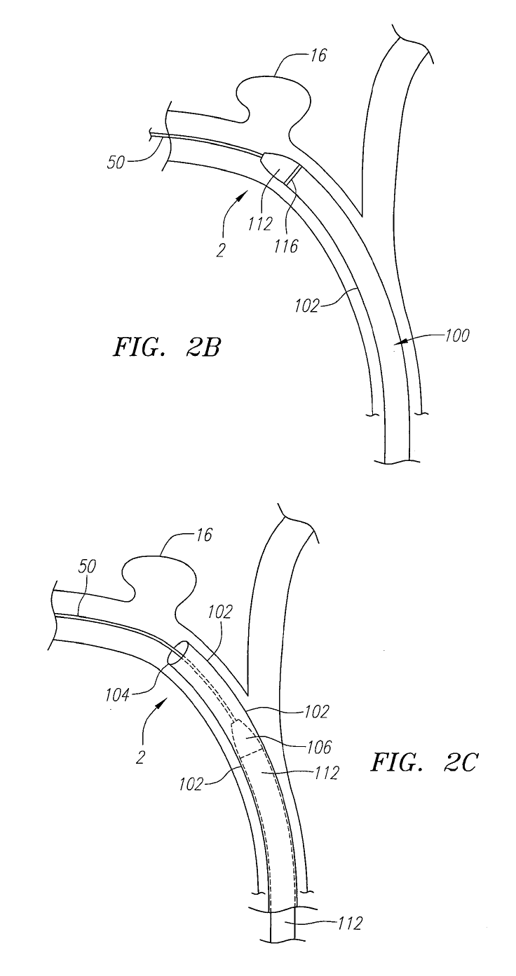

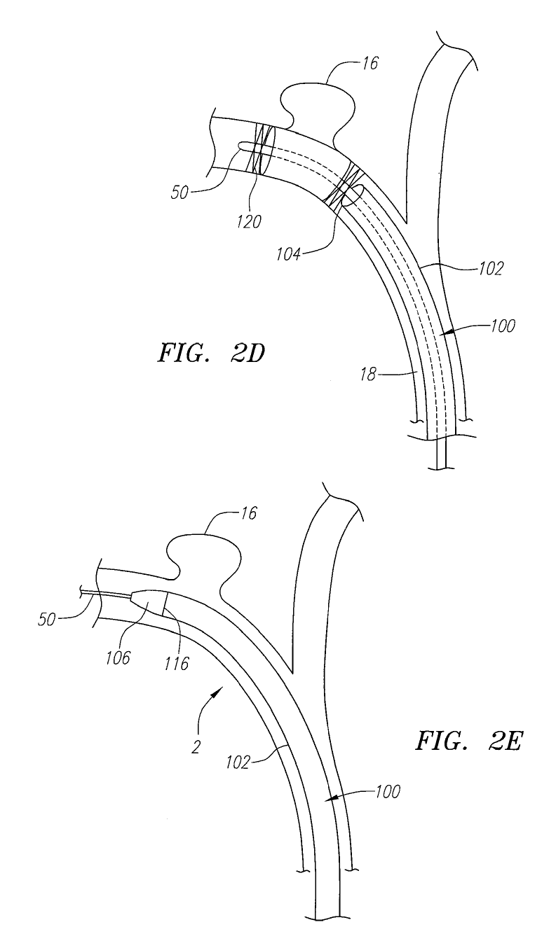

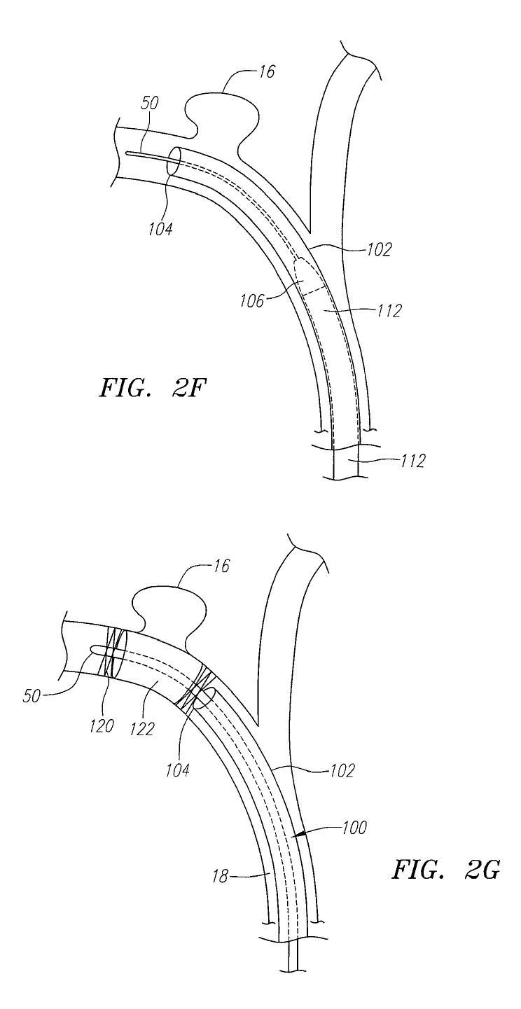

FIG. 2A shows system 100 when advanced into a remote region 2 of the vasculature. As illustrated, the remote region is within the vasculature of the brain 10. Systems so-suited for use in the vasculature of the brain must have the requisite flexibility for navigating the tortuous path into the region, and at the same time have sufficient column and/or torsional strength to be advanced and manipulated from an external access site, often via the femoral artery access at the groin. The system requires coupling and co-delivery of the core member and the sleeve in order to navigate to and place the sleeve at remote vasculature. The sleeve does not possess sufficient column strength to navigate the vasculature on its own. The system of tandem navigation of core and sleeve allows for minimizing the wall thickness of tubular sleeve 102 which in turn allows for a sleeve design having a larger bore or lumen internal diameter (ID) for a given outer/outside diameter (OD). This larger lumen diameter is then made available as an access lumen to the target site once the core is removed from the sleeve. Once cleared of the core, the large sleeve lumen (advantageously having an ID of between about 0.050 and 0.070 inches) allows for delivery of therapeutic devices or agents that would not otherwise be deliverable using a standard microcatheter, making device-assisted treatment of cerebral vasculature possible. Still, it is to be appreciated that the subject approach may also find use in cases where the ID is between about 0.025 and about 0.050 inches.

FIG. 2A shows distal end of system 100 adjacent aneurysm 16 in vessel 18 lateral wall. FIG. 2B shows a magnified view of site 2 in the cerebral vasculature of FIG. 2A. To access the site, a physician advances a guidewire 50 to site 2 in coordinated action with system 100 and possibly other catheters in “telescoping” fashion as commonly done by skilled physicians. Otherwise, the physician first positions the guidewire then advances system 100 (having core 112 and sleeve 102) along guidewire 50 to, or adjacent to, site 2. Again, the wire may be used in conjunction with such other guide catheters as commonly used, for example, in more tortuous anatomy, the wire can be advanced some distance, and then the catheter advanced over the wire. While not shown, a stiff large-lumen guide-catheter can also be employed to support the wire and catheter system 100 in more proximal anatomy.

Preferably, the outside diameter of the sleeve is hydrophylically coated as is standard practice with microcatheters to assist their passage in the vascular space, and/or through other co-axial catheters(s). Also it is to be appreciated that either one or both of the members 102/112 may comprise a layer of lubricious material (e.g. PTFE) along their walls.

As discussed above, the location of core tip 106 and/or sleeve and can be identified using a radiopaque marker(s) via medical imaging. Once the physician is satisfied with placement of sleeve tip 104, core member 112 is withdrawn as shown by FIG. 2C.

Optionally, guidewire 50 can remain at the site, may be withdrawn with core member 112, or may be withdrawn subsequent to withdrawal of core member 112. A physician's choice in this regard will likely depend on the nature of the implant intended for delivery. When using the system to extend the range of over-the-wire (or rapid exchange deliverable) stent-grafts, leaving the wire in can give more support to help tracking through the sleeve. Guidewire 50 will be removed when the catheter is used to deliver an implant mounted to a delivery device with no guidewire lumen.

FIG. 2D shows delivery of stent 120 to site 2 using delivery device 100. Stent 120 is positioned at aneurysm 16 over wire 50 after passage through sleeve 102 through distal opening 104 in vessel 18. In this case, a balloon-expandable stent-graft (such as shown in provisional patent application U.S. Ser. No. 61/035328 filed Mar. 10, 2008) is implanted.

FIGS. 2E, 2F and 2G show (in sequence) positioning of the subject device to deliver a self-expanding stent-graft. FIG. 2E depicts delivery of device 100 to site 2 having aneurysm 16 in the neurovasculature. Atraumatic tip 106 (of core 112) is adjacent sleeve 102 at marker 116. As depicted in FIG. 2F, upon withdrawal of core 112 having tip 106, sleeve 102 rests in vessel 18 and remains at the site distal to aneurysm 16. As depicted in FIG. 2G, sleeve 102 provides access over wire 50 to stent-graft 122 which can then be delivered to cover the neck of aneurysm 16 at the target treatment site upon withdrawal of sleeve relative to a pusher (not shown) thereon to expose the stent as show in FIG. 26.

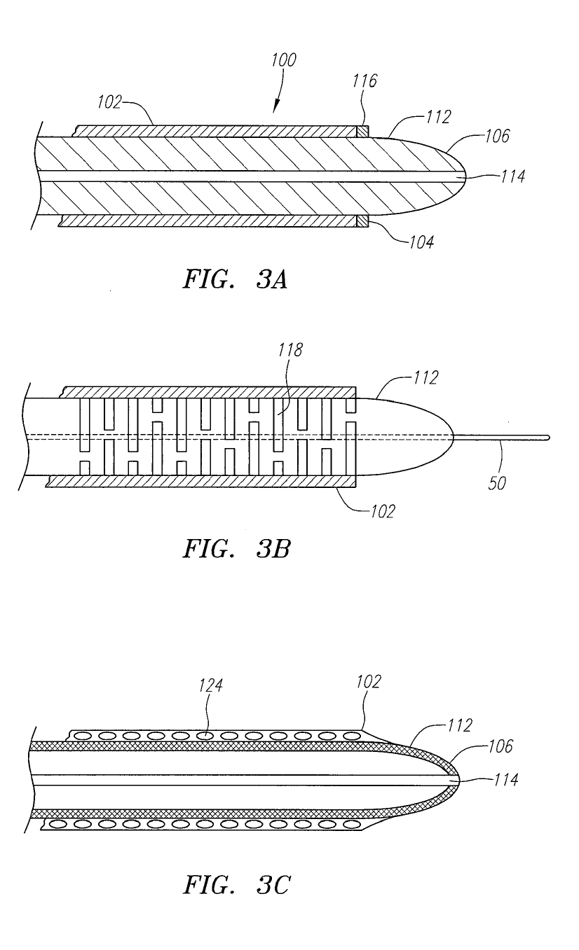

Regarding further structural details of the subject devices, FIG. 3A shows a partial cross sectional view of tubular sleeve 102, with core member 112 received therein, with atraumatic tip 106 extending from opening 104 of core 112. In one variation, sleeve 102 comprises a material selected from PTFE, PEEK, HDPE, PET, or a combination thereof. However, the sleeve can be fabricated from any medical grade polymer.

In the variation shown in FIG. 3A, core 112 comprises a solid structure having a guide wire lumen 114 extending therethrough. Variations of core 112 can be fabricated from or in combination with PEEK, PTFE, PET, HDPE, NiTi, CoCr, and stainless steel. The polymeric materials may be employed in metallic reinforced (e.g. braid, modified cable, coil, etc.) laminate or composite. Hollow cable (e.g. NiTi or stainless steel) such as available through Asahi Metals of Osaka, Japan and Fort Wayne Metals of Fort Wayne, Ind. may be used for the core. A metal core can in addition, be coated in polymer to improve its lubricity and/or fit within sleeve 102.

Generally speaking, typical catheter wall construction technologies (described in U.S. Pat. No. 5,658,264, U.S. Pat. No. 5,702,373, U.S. Pat. No. 4,739,768, and U.S. Pat. No. 5,851,203 which are incorporated herein by reference) can be used to construct the various elements of the microcatheter system subject to the limitations herein. Although, the device may accommodate any size guidewire, typical guidewire sizes required to reach remote vascular sites include guidewires having diameters ranging from 0.010 inches to 0.018 inches. Guidewire lumen within core 112 is sized to accommodate the appropriate guidewire accordingly. In preferred embodiments, the core lumen that accommodates the guidewire will be about 0.20 inches (to accommodate an 0.018 inch guidewire), or about 0.016 inches (to accommodate an 0.014 inch guidewire) or about 0.012 inches (to accommodate an 0.010 inch guidewire) as distinguished from typical neurovascular microcatheters that have larger working lumens (of 0.021 and 0.027 inches, respectively).

FIG. 3B illustrates another variation of core member 112 having a series of slots, cuts, or grooves 118 that increase its flexibility. Slots 118 can extend in a pattern uniformly along the length of core 112 from core tip to core hub (not shown). Alternatively, slots 118 can extend over a portion of core 112 to vary stiffness along the core length. Preferably, core 112 is stiffer at the proximal end of the microcatheter and more flexible (less stiff) at or toward the distal end. Stated otherwise, optimally, the core will be stiffer at the proximal end, and move on its length to increased flexibility distally, resolving in a highly flexible segment at the distal end. At the very distal end, or tip, the core member can be quite flexible for a very brief section at the atraumatic tip to facilitate maximal protection of the vessels being accessed.

Embodiments that include such modifications as slots can have the slots circumferentially spaced about the core member as shown in FIG. 3B, or the slots may comprise one or more helical slots that extend over a portion or length of the core member (not shown). Although any configuration of slots is possible, the slots in the illustrated variation are placed in a manner similar to that shown in U.S. Pat. No. 6,482,489 assigned to Precision Vascular Systems, Inc. and incorporated by reference herein. Embodiments of the catheter system that have a variable stiffness in the core can also be achieved by varying the pitch at which a wire or group of wires is wound to form a hollow cable core. So too, the number of wires incorporated in the cable may decrease distally. Other standard means to vary the stiffness (such as proximally jacketing the core) may additionally (or alternatively) be employed.

FIG. 3C illustrates another variation of tubular sleeve 102 including a reinforcing structure 124 in the wall of the sleeve. The reinforcing structure can comprise a braid, winding, coil, ring, or any such structure. When incorporated in sleeve 124, such structure will primarily be incorporated to avoid catheter kinking and/or ovalization in tortuous vasculature when the core member is removed. The reinforcing structure may comprise a ribbon or wire and may be selected from a super elastic alloy or a spring steel material. Examples of such reinforcement are found in U.S. Pat. No. 5,658,264 and U.S. Pat. No. 5,702,373, both to Samson, the entirety of each of which is incorporated by reference herein. FIG. 3C also illustrates an embodiment of core 112 comprising multi-filament hollow cable.

Variations in thickness and ultimately stiffness and flexibility of the sleeve can also be built into the design elements of system 100. Typical and optimal configuration of sleeve 102 can include an inner lining of PTFE (to encourage lubricity with the core member) followed by a braid, followed by a layer of Pebax™ of varying stiffness on the outside of the sleeve. For example, the proximal end of the composite laminate sleeve can be stiffer by virtue of a durometer of 75D of the Pebax™ extending for about 120 cm of the sleeve length. The proximal region of the sleeve can be followed by a space of transitional durometer (about 55D) of about 30 cm in length, followed by a distal region having a flexibility of about 35D at the end. At the very tip, a region of the sleeve extending only about 2 mm can be without braid or other reinforcing material, thus being made of just PTFE and Pebax™.

Preferably, core member diameter will be matched to the inner diameter of the sleeve to permit advancement of interlocked core/sleeve unit as well as later withdrawal of the core. By “matched”, what is meant that they are sized to permit both tandem advancement of the core and sleeve in the vasculature, as well as eventual removal (withdrawal) of the core from the sleeve. Typically, to facilitate retraction, the core is undersized relative to the sleeve lumen ID by between about 0.001 and about 0.005 inches. However, a greater size differential is also possible. Conversely, so the sleeve/core members (102/112) track well together, a distal interference fit (e.g., by a reduced diameter distal end 106 of sleeve 102) may be advantageous. In other words, the core can “hug” the sleeve at the distal end (relative to its interference with the sleeve at the proximal end) so that the navigatability at the tip of the microcatheter is optimized.

The subject methods may include each of the physician activities associated with implant positioning and release. As such, methodology implicit to the positioning and deployment of an implant device forms part of the invention. Such methodology may include placing a stent or stent-graft implant at the opening of a brain aneurysm, introducing embolic coils to an aneurysm prior to placing an implant at the opening to seal the aneurysm, introducing a neuro-embolic braid ball as described in provisional patent application U.S. Ser. No. 61/046,384 filed Apr. 18, 2008 or other applications. In some methods, the various acts of implant introduction to an aneurysm are considered.

More particularly, a number of methods according to the present invention involve the manner in which the core/sleeve delivery system operates in reaching a treatment site, for example. Other methods concern the manner in which the system is prepared for delivering an implant (after placement of the sleeve and removal of the core). For example, methods include stenting a body passageway by locating the guidewire within the sleeve at a site within the body passageway, introducing a delivery catheter onto the guidewire under circumstances in which the stent is held open to receive a guidewire, and feeding a delivery catheter over or along the guidewire within the already in place sleeve once its working lumen is cleared. Any method herein may be carried out in any order of the recited events which is logically possible, as well as in the recited order of events, or slight modifications of those events or the event order.

Also, it is contemplated that any optional feature of the inventive variations described may be set forth and claimed independently, or in combination with any one or more of the features described herein. Reference to a singular item, includes the possibility that there is a plurality of the same items present. More specifically, as used herein and in the appended claims, the singular forms “a,” “an,” “said,” and “the” include plural referents unless specifically stated otherwise. In other words, use of the articles allow for “at least one” of the subject item in the description above as well as the claims below. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in the claims shall allow for the inclusion of any additional element irrespective of whether a given number of elements are enumerated in the claim, or the addition of a feature could be regarded as transforming the nature of an element set forth in the claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of the claim language.

All references cited are incorporated by reference in their entirety. Although the foregoing invention has been described in detail for purposes of clarity of understanding, it is contemplated that certain modifications may be practiced within the scope of the appended claims.

1. A microcatheter system comprising:
a tubular sleeve including a proximal portion, a distal portion and a body lumen extending therethrough, the distal portion terminating at a distal opening;
a core member removably located within the body lumen, the core member having a proximal end, a distal end terminating in an atraumatic tip and a guidewire lumen extending therethrough, where a length of the core member is greater than a length of the tubular sleeve and a flexibility of the core member permits navigation to the cerebral vasculature; and
where the tubular sleeve comprises a length, outer diameter and flexibility to permit navigation to the cerebral vasculature only when the core member is located within the body lumen.

2. The microcatheter system of claim 1, wherein the tubular sleeve has a wall with a thickness between about 0.003 inches and about 0.005 inches.

3. The microcatheter system of claim 1, the core member comprising a hollow cable.

4. The microcatheter system of claim 1, the core member comprising a pattern having alternating filled and open regions, the pattern designed to increase the flexibility of the core member.

5. The microcatheter system of claim 3, wherein the core member is metallic.

6. The microcatheter system of claim 3, wherein the core member includes a polymeric coating.

7. The microcatheter system of claim 1, wherein the tubular sleeve comprises a reinforcing structure over a polymeric layer.

8. The microcatheter system of claim 7, wherein the polymeric layer is PTFE.

9. The microcatheter system of claim 7, wherein the tubular sleeve comprises inner and outer polymeric layers.

10. The microcatheter system of claim 7, where the reinforcing structure is selected from braid and winding.

11. The microcatheter system of claim 1, wherein the atraumatic tip comprises a bullet shape.

12. The microcatheter system of claim 1, wherein a proximal portion of the tubular sleeve comprises a sleeve hub and the proximal end of the core member comprises a core hub, where the core hub and sleeve hub are lockable together for delivery of the microcatheter.

13. The microcatheter system of claim 1, wherein the core lumen is at least about 0.050 inches.

14. A method of vascular treatment, the method comprising:
advancing a guidewire to a remote region in a blood vessel of cerebral vasculature,
navigating a microcatheter over the guidewire to the blood vessel, where the microcatheter comprises a tubular sleeve having a core member located within a lumen of the tubular sleeve, the core member having an atraumatic tip extending distally beyond a distal opening of the tubular sleeve, where without the core member the tubular sleeve lacks sufficient column strength to advance to the remote region in the blood vessel of cerebral vasculature; and,
withdrawing the core member from the tubular sleeve while maintaining the tubular sleeve at the remote region of the accessed blood vessel.

15. The method of claim 14, further comprising advancing a delivery system having an implant through the tubular sleeve to the remote region in a blood vessel of the cerebral vasculature, and deploying the implant in the blood vessel.

16. The method of claim 15, where the core member and guidewire are withdrawn together.
